(19) United States
(12) Patent Application Publication
(10) Pub. No.: US 2021/0393722 A1
(43) Pub. Date: Dec. 23, 2021

(54) COMPOSITION FOR IMPROVING SKIN BARRIER DAMAGE AND/OR ALLEVIATING SKIN INFLAMMATION, CONTAINING 3,5-DICAFFEOYLQUINIC ACID AS ACTIVE INGREDIENT

(71) Applicants: KOREA UNIVERSITY RESEARCH AND BUSINESS FOUNDATION, Seoul (KR); KOREA INSTITUTE OF SCIENCE AND TECHNOLOGY, Seoul (KR)

(72) Inventors: Hong Seog SEO, Seoul (KR); Yong Jik LEE, Incheon (KR); Hyoung Ja KIM, Seoul (KR); Chang Bae JIN, Seoul (KR)

(73) Assignees: KOREA UNIVERSITY RESEARCH AND BUSINESS FOUNDATION, Seoul (KR); KOREA INSTITUTE OF SCIENCE AND TECHNOLOGY, Seoul (KR)

(21) Appl. No.: 17/288,632

(22) PCT Filed: Oct. 31, 2019
(86) PCT No.: PCT/KR2019/014547

§ 371 Date: Apr. 26, 2021

(30) Foreign Application Priority Data
Oct. 31, 2018 (KR) 10-2018-0132216

(51) Int. Cl. A61K 36/28; A61K 31/216; A23L 33/10

(52) U.S. Cl. CPC A61K 36/28; A61K 31/216; A61K 2236/39; A61K 2236/333; A23L 33/10

(57) ABSTRACT

The present invention relates to a composition for improving skin barrier damage and/or alleviating skin inflammation, wherein 3,5-dicaffeoylquinic acid isolated from an Aster glehni (AG) extract is introduced as an active ingredient of a cosmetic composition or a pharmaceutical composition for improving skim barrier damage and alleviating skin inflammation, thereby being effective in preventing and improving atopic dermatitis.

### TECHNICAL FIELD

The present invention relates to a composition for improving skin barrier damage and/or alleviating skin inflammation, more particularly to a composition for improving skin barrier damage and/or alleviating skin inflammation, which contains 3,5-dicaffeoylquinic acid as an active ingredient.

### BACKGROUND ART

Skin is composed of three layers, the epidermis, the dermis and the subcutaneous tissue (hypodermis). In particular, the epidermis is the outermost region of the skin and can be subdivided into four layers, stratum basale, stratum spinosum, stratum granulosum and stratum corneum (the outermost thin layer of the skin). Keratinocytes are the major cells constituting the epidermis and contribute to the formation of a defensive barrier for the body through keratinization. In the stratum spinosum, the keratinocytes produce keratins 1 and 10. Keratin 10 is the major component of a desmosome. In general, a skin barrier refers to the stratum corneum, and it is composed of keratinocytes, cornified envelopes, corneal epithelial cells and intercorneocyte lipids. The cornified envelope is composed of transglutaminase, involucrin, loricrin, and filaggrin, and serves as a skin barrier by forming a multilayered structure of intercorneocyte lipids. The intercorneocyte lipids include ceramides, cholesterols, free fatty acids and cholesterol sulfates. Among them, the content of the ceramides is the highest. The main function of the skin barrier is to prevent the loss of body fluids, attack by toxins and invasion of pathogen (Department of Dermatology, Faculty of Medicine and Graduate School of Medicine Hokkaido University. Shimizu's Textbook of Dermatology. http://www.derm-hokudai.jp/shimizu-dermatology/ch01 (13.02.2017); H. H. Jang, S. N. Lee, Asian J Beauty Cosmetol, 14, 339, 2016). Accordingly, damage to the skin barrier may cause not only the simple mechanical failure of the outer layer of the skin but also severe immune responses due to invasion of various pathogens. In general, the anionic surfactant sodium dodecyl sulfate (SDS) induces not only skin irritation but also the polarization of skin surface, disruption of the skin barrier and extraction of epidermal lipids. In addition, SDS increases transepidermal water loss from the stratum corneum and induces inflammation.

Atopic dermatitis (AD) occurs not only in early childhood but also in adults. In general, AD is a chronic, itchy, inflammatory skin disease characterized by increased immunoglobulin E (IgE) production, recurrence of eczematous skin lesions, infiltration of inflammatory immune cells and abnormalities in the skin barrier. The primary cause is unknown and the occurrence of AD is increasing with the progress of industrialization. Therefore, it is very urgent and important socially and economically to find an effective therapy for AD. 2,4-Dinitrochlorobenzene (DNCB) is known as a representative material that induces contact dermatitis and AD (Medscape. Atopic Dermatitis. http://emedicine.medscape.com/article/1049085-overview#a4(15.02.2017); T. Bieber, N Engl J Med., 358, 1483, 2008).

Aster glehni (AG) has been used in Korean traditional medicine to treat fever, pain, phlegm and cough. Other effects of AG were also reported previously. The ethyl acetate extract of AG inhibited the expression of tyrosinase and tyrosinase-related protein 1, which are involved in melanin biosynthesis. In another study, the ethyl acetate extract of AG has been reported to have the effect of inhibiting the expression of inducible nitric oxide synthase (iNOS), which is involved in antioxidant effect and inflammation (Y. Fujii et al., Skin Pharmacol Physiol., 22 240, 2009).

Meanwhile, it is not known that 3,5-dicaffeoylquinic acid isolated from the Aster glehni (AG) extract has an effect of improving skin barrier damage or alleviating skin inflammation.

The inventors of The present invention have made consistent efforts to find out a composition having an effect of improving skin barrier damage and/or alleviating skin inflammation for prevention and treatment of diseases such as atopy. As a result, they have found out that 3,5-dicaffeoylquinic acid isolated from the Aster glehni (AG) extract has superior effect of improving skin barrier damage and/or alleviating skin inflammation and completed The present invention.

### DISCLOSURE

### Technical Problem

The present invention is directed to providing a pharmaceutical composition for improving skin barrier damage and/or alleviating skin inflammation, which contains 3,5-dicaffeoylquinic acid as an active ingredient.

The present invention is also directed to providing a composition for external application containing 3,5-dicaffeoylquinic acid as an active ingredient, which is in the form of a liquid, an ointment, a cream, a lotion, a spray, a patch, a gel or an aerosol.

The present invention is also directed to providing a cosmetic composition for improving skin barrier damage and/or alleviating skin inflammation, which contains 3,5-dicaffeoylquinic acid as an active ingredient.

The present invention is also directed to providing a functional health food composition for improving skin barrier damage and/or alleviating skin inflammation, which contains 3,5-dicaffeoylquinic acid as an active ingredient.

### Technical Solution

To achieve said purposes, the present invention provides a pharmaceutical composition for improving skin barrier damage and/or alleviating skin inflammation, which contains 3,5-dicaffeoylquinic acid as an active ingredient.

The present invention also provides a composition for external application containing 3,5-dicaffeoylquinic acid as an active ingredient, which is in the form of a liquid, an ointment, a cream, a lotion, a spray, a patch, a gel or an aerosol.

The present invention also provides a cosmetic composition for improving skin barrier damage and/or alleviating skin inflammation, which contains 3,5-dicaffeoylquinic acid as an active ingredient.

The present invention also provides a functional health food composition for improving skin barrier damage and/or alleviating skin inflammation, which contains 3,5-dicaffeoylquinic acid as an active ingredient.

The present invention also provides a method for preventing or treating skin barrier damage and/or skin inflammation, which includes a step of administering 3,5-dicaffeoylquinic acid to a subject.

The present invention also provides a use of 3,5-dicaffeoylquinic acid for preparation of a medication for preventing or treating skin barrier damage and/or skin inflammation.

In an exemplary embodiment of the present invention, the 3,5-dicaffeoylquinic acid may be one isolated from an Aster glehni (AG) extract.

In another exemplary embodiment of the present invention, the 3,5-dicaffeoylquinic acid may be one obtained from an ethyl acetate fraction of an Aster glehni (AG) extract.

### Advantageous Effects

3,5-Dicaffeoylquinic acid of the present invention has no side effect because it is isolated from a natural product. When the compound is introduced as an active ingredient of a cosmetic composition or a pharmaceutical composition, it is effective in preventing, treating and improving inflammatory skin diseases such as atopic dermatitis, etc. by improving skin barrier damage and alleviating skin inflammation.

### BRIEF DESCRIPTION OF DRAWINGS

FIG. 1 shows the HPLC analysis result of the ethyl acetate extract from Aster glehni (AG). (1) 5-CQA: 5-caffeoylquinic acid, (2) 3,4-DCQA: 3,4-dicaffeoylquinic acid, (3) 3,5-epi-DCQA: 3,5-epi-dicaffeoylquinic acid, (4) 3,5-DCQA: 3,5-dicaffeoylquinic acid, (5) 4,5-DCQA: 4,5-dicaffeoylquinic acid, (6) 3,5-DCQA-Me: methyl 3,5-dicaffeoylquinate, (7) 4,5-DCQA-Me: methyl 4,5-dicaffeoylquinate.

FIG. 2A and FIG. 2B show the western blot analysis result for PPARδ, AMPK and SPTLC2 in HaCaT cells treated with an Aster glehni extract, SDS, DNCB or GSK0660 (Ctrl: untreated control group, DNCB: group treated with DNCB, D+AG: group treated with DNCB and A, D+A+GSK: group treated with DNCB, AG and GSK0660, SDS: group treated with SDS, S+AG: group treated with SDS and AG, S+A+GSK: group treated with SDS, AG and GSK0660).

FIGS. 3A-3D show the immunocytochemical analysis result for keratin, involucrin, defensin and TNFα in HaCaT cells treated with an Aster glehni extract, SDS, DNCB or GSK0660 (Ctrl: untreated control group, DNCB: group treated with DNCB, D+AG: group treated with DNCB and A, D+A+GSK: group treated with DNCB, AG and GSK0660, SDS: group treated with SDS, S+AG: group treated with SDS and AG, S+A+GSK: group treated with SDS, AG and GSK0660).

FIG. 4A and FIG. 4B show the immunohistochemical staining result for keratin, involucrin, defensin, and TNFα in HaCaT cells treated with a TRPV4 or AMPK antagonist.

FIG. 5 shows the western blot analysis result for TRPV4, AMPK, PPARδ and SPTLC2 in HaCaT cells treated with 3,5-DCQA.

FIG. 6A and FIG. 6B show the immunohistochemical staining result for keratin, involucrin, defensin and TNFα in HaCaT cells treated with 3,5-DCQA.

FIG. 7 schematically shows the functional mechanism of an Aster glehni (AG) extract in keratinocytes (arrows mean activation, horizontal line means inhibition, and double vertical line means blocking).

### BEST MODE

In the present invention, it was confirmed that the cells treated with 3,5-dicaffeoylquinic acid show increased expression of PPARδ, phosphorylated AMPK, SPTLC2, keratin, involucrin and defensin proteins as compared to control group cells treated with SDS or DNCB, and that the increased TNFα expression in the cell group treated with SDS or DNCB only was decreased in the cell group to which 3,5-dicaffeoylquinic acid isolated from an Aster glehni (AG) extract was further added. The order of action of each biomarker in the AG pathway was identified as TRPV4→PPARδ→AMPK from antagonist treatment, and it was confirmed that 3,5-dicaffeoylquinic acid isolated from an Aster glehni (AG) extract can ameliorate the injury of keratinocytes caused by SDS or DNCB through the sequential regulation of the TRPV4→PPARδ→AMPK pathway.

Accordingly, in an aspect, the present invention relates to a pharmaceutical composition for improving skin barrier damage and/or alleviating skin inflammation, which contains 3,5-dicaffeoylquinic acid isolated from an Aster glehni (AG) extract as an active ingredient.

Chemical Formula 1 is the structural formula of 3,5-dicaffeoylquinic acid.

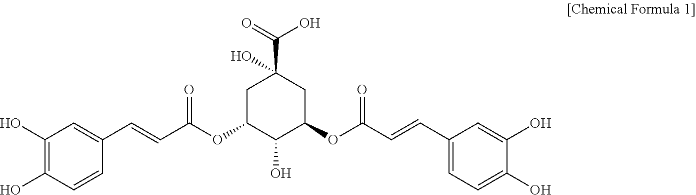 [SMILES: O=C(/C=C/c1ccc(O)c(O)c1)O[C@@H]1C[C@](O)(C(=O)O)C[C@@H](OC(=O)/C=C/c2ccc(O)c(O)c2)[C@H]1O]

The term “skin barrier” used in the present invention refers to the stratum corneum, which is the outermost skin layer consisting of keratinocytes. It is the most important primary defense line against toxic substances, microorganisms, mechanical stimulations or UV, and serves the function of providing an environment for normal function of skin by preventing the loss of electrolytes or water through the skin.

The term “improving skin barrier damage” used in the present invention refers to preventing or improving skin barrier damage by enhancing the barrier function of the stratum corneum which is the outermost layer of the skin.

Regarding the function of treating and improving skin barrier damage according to the present invention, the stratum corneum which is the outermost layer of the epidermis is mostly composed of flat corneocytes without nuclei. The multilamellar lipid layer formed of intercellular lipids such as ceramides, cholesterols and fatty acids synthesized by the corneocytes of the skin barrier, which is maintained through normal division and differentiation of epidermal cells, serves as a defensive barrier of preventing evaporation of water in the skin. Among the intercellular lipids, ω-hydroxyceramide is covalently bonded to involucrin, which is a protein in the outer layer of corneocytes, thereby forming a corneocyte lipid envelope (CLE). This treats and improves barrier damage by physically stabilizing the intercellular lipids of the multilamellar lipid layer.

When applied to skin, the composition of the present invention is delivered to the stratum corneum and exhibits an effect of improving the thickness of the epidermal layer and restoring skin barrier damage by promoting the differentiation of keratinocytes. Therefore, it can be usefully used for treatment and prevention of a skin disease caused by skin barrier damage. The skin disease caused by skin barrier damage includes atopic dermatitis, xeroderma, psoriasis, ichthyosis, acne, etc., although not being limited thereto.

In the present invention, the term “improving skin barrier damage” includes protection of skin, improvement of skin condition, alleviation of inflammatory response of the skin, improvement of immunological diseases, improvement of skin barrier function, alleviation of skin irritation, proliferation and regeneration of skin cells, antioxidation ability, enhancement of collagen synthesis, etc.

In the present invention, “alleviating skin inflammation” refers to improvement and treatment of skin troubles such as skin inflammation, itchiness, etc.

In the present invention, “alleviating inflammation” means inhibition of inflammation, and the inflammation refers to one of defensive responses of the biological tissue to stimuli, including degeneration, circulatory disorder, exudation and tissue proliferation. More specifically, the inflammation is part of innate immunity. As in other animals, human innate immunity recognizes the patterns of cell surfaces that are specific to pathogens. Phagocytes recognize the cells having such surfaces as nonself cells and attack the pathogens. Inflammatory response occurs when the pathogens invade through the physical barrier of the body. The inflammatory response is a nonspecific defensive action of creating a hostile environment against the microorganisms that have invaded the wound site. In the inflammatory response, when a scar is made or an external pathogen has entered the body, leukocytes responsible for immune response in the early stage are recruited and express cytokines. Accordingly, the intracellular expression level of cytokines becomes an index of the activation of inflammatory response.

Examples of the skin disease related with inflammation include atopic dermatitis, psoriasis, erythematous diseases caused by radiation, chemicals, burn, etc., acid burn, bullous dermatosis, lichen, allergic itchiness, seborrheic eczema, acne rosacea, pemphigus vulgaris, erythema exsudativum multiforme, erythema nodosum, balanitis, vulvitis, inflammatory hair loss such as alopecia areata, cutaneous T cell lymphoma, etc., although not being limited thereto.

The term “prevention” used in the present invention refers to any action of inhibiting skin barrier damage or inflammatory response or delaying the onset of a disease caused thereby by administering the pharmaceutical composition according to The present invention.

The term “treatment” used in the present invention refers to any action of improving or favorably changing the symptom of a skin disease caused by skin barrier damage or inflammatory response by administering the pharmaceutical composition according to The present invention.

The term “improvement” used in the present invention refers to any action of decreasing the parameters associated with a state to be treated, e.g., the severity of a symptom.

In the present invention, the term “extract” refers to a substance obtained by isolating from Aster glehni (AG).

In the present invention, the Aster glehni (AG) extract may be one extracted with one or more extraction solvent selected from a C₁₋₄ alcohol, an aqueous solution containing a C₁₋₄ alcohol, dichloromethane, acetone and an aqueous acetone solution.

In the present invention, the Aster glehni (AG) extract may be a fractional extract further fractionated with any one selected from a group consisting of ethyl acetate, hexane, chloroform and butanol.

The extraction may be performed by an extraction method known in the art, e.g., cold precipitation, hot water extraction, ultrasonic extraction, reflux condensation extraction, etc., although not being limited thereto. Extraction temperature may be adequately selected by those skilled in the art depending on the extraction method. For example, the extraction may be performed at 20-100° C., although not being limited thereto. In addition, extraction time varies depending on the extraction method and adequate extraction time may be selected by those skilled in the art. The extraction may be performed once or several times, within from about 1 hour to 10 days. Specifically, the extraction may be performed 2-3 times at room temperature for about 2 days using the extraction solvent described above.

In the present invention, the composition may increase the expression of TRPV (transient receptor potential cation channel subfamily V member 4), PPAR-δ (peroxisome proliferator-activated receptor delta) and AMPK (5′ AMP-activated protein kinase).

In the present invention, PPARδ and AMPK are involved in cell survival and antiinflammatory response. Ceramides are important lipid components of the skin barrier, and serine palmitoyltransferase is an enzyme which catalyzes the rate-limiting step of ceramide biosynthesis. And, TRPV4 is known to be involved in the formation of intercellular junctions in keratinocytes. SPTLC2 is the long chain base subunit of serine palmitoyltransferase.

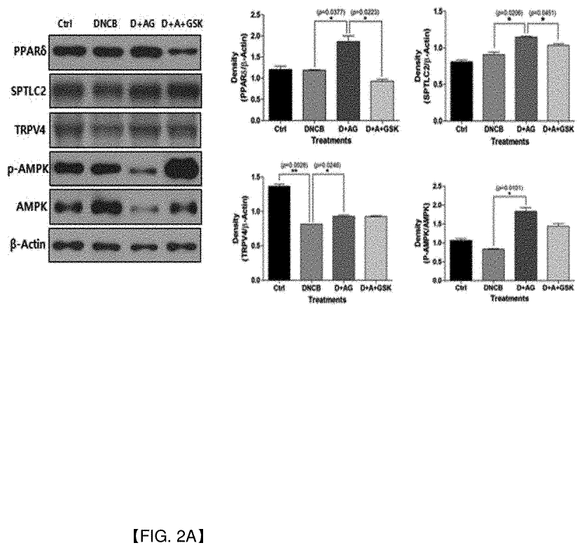

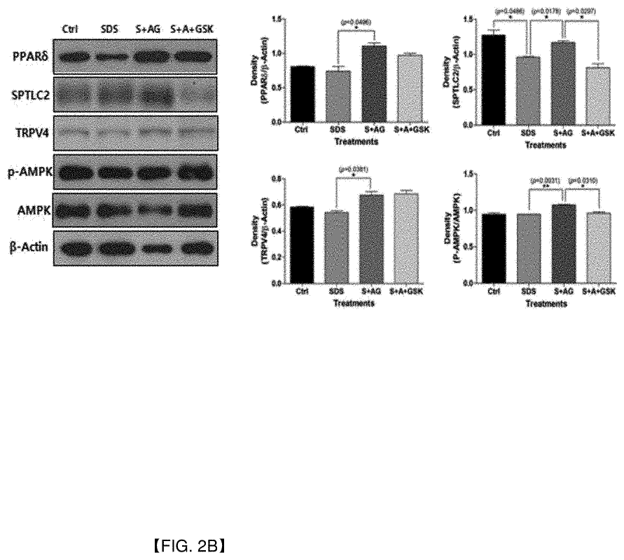

In an example of the present invention, the expression level of the PPARδ, AMPK and SPTLC2 proteins in HaCaT cells treated with AG together with SDS or DNCB was measured. As a result, it was confirmed that an AG extract increases the expression of the PPARδ, P-AMPK, SPTLC2 and TRPV4 proteins as compared to the cell group treated only with DNCB and SDS (FIG. 2A and FIG. 2B).

In the present invention, the composition may decrease the expression of TNF-α (tumor necrosis factor-alpha).

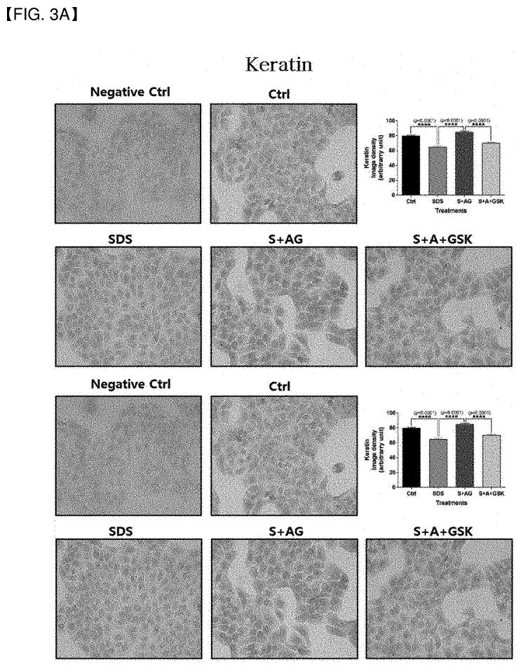

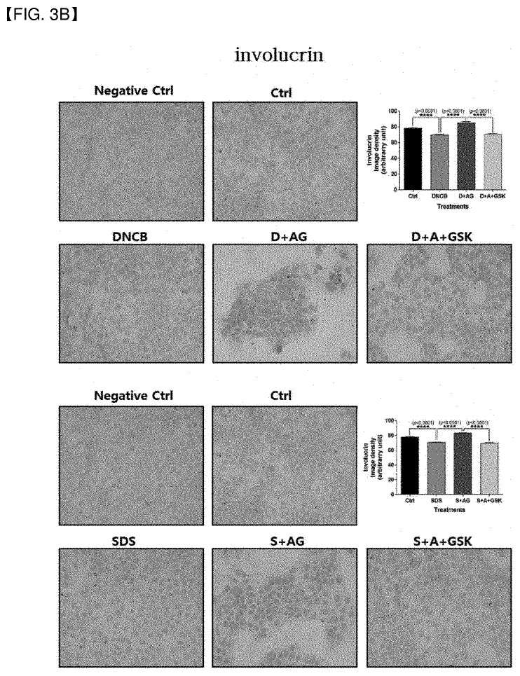

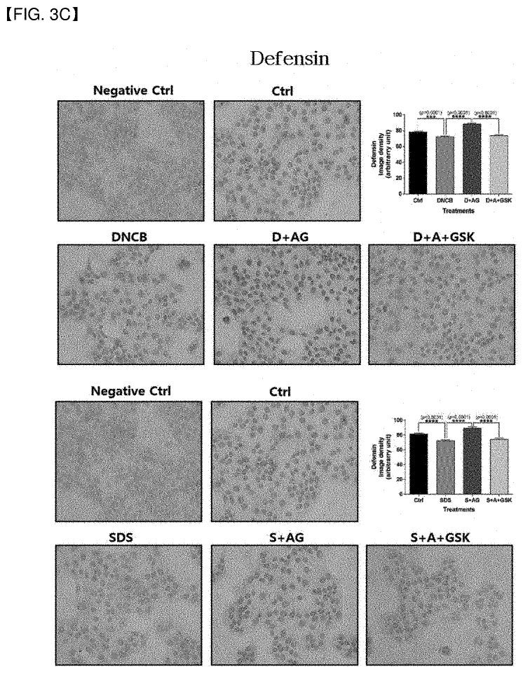

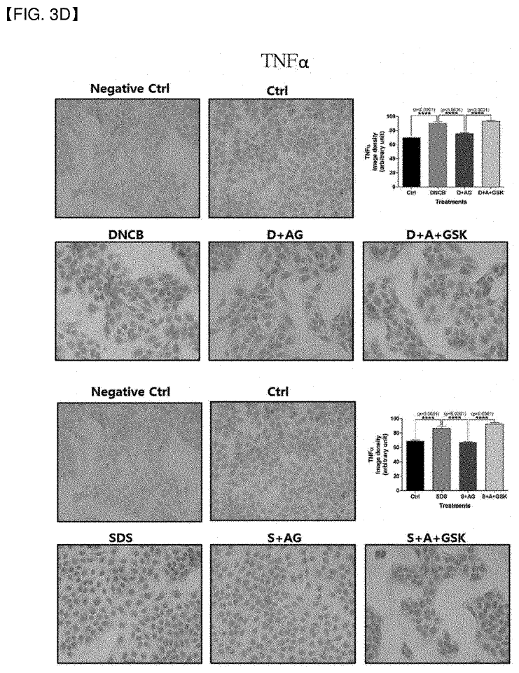

In another example of the present invention, it was confirmed that the expression of TNFα increased in the cell group treated only with DNCB or SDS was decreased in the cell group to which an AG extract was further added (FIG. 3D).

In an example of the present invention, the expression level of biomarkers related with the maintenance of skin barrier in HaCaT cells treated with SDS or DNCB was measured to clarify the effect of AG on the protection of the skin barrier and the mechanism thereof. In particular, it was examined whether the expression of peroxisome proliferator-activated receptor δ (PPARδ), AMP-activated protein kinase (AMPK) and transient receptor potential cation channel subfamily V member 4 (TRPV4) plays an important role in the functional mechanism of skin protection by AG.

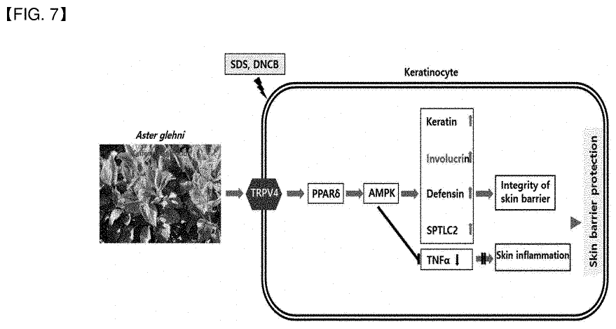

As a result, it was confirmed that the caffeoylquinic acid-rich AG extract can protect the skin barrier through the sequential regulation of the TRPV4-PPARδ-AMPK pathway in human keratinocytes HaCaT cells treated with SDS or DNCB, and that the skin barrier is protected through antiinflammatory action owing to inhibition of TNFα (FIG. 7).

In the present invention, defensins are antibacterial, antimicrobial and antiviral small cationic peptides produced by various cell types, and includes three subfamilies of α, β and θ defensins. β-Defensin is mainly secreted by keratinocytes.

In the present invention, PPARδ, which is a nuclear receptor, alleviates metabolic diseases such as obesity and atherosclerosis. The skin barrier-protecting effect of PPARδ has been studied as follows: PPARδ ligand treatment stimulates stratum corneum formation and permeability barrier development in a fetal rat explant culture model, and the epidermal integrity is disrupted and inflammation is increased in PPARδ knockout mice. Generally, AMPK is involved in decreasing inflammation as well as ameliorating the symptoms of metabolic disease and is regulated by PPARδ. Activated AMPK suppresses the increase of matrix metalloproteinase 1 (MMP1) induced by ultraviolet radiation in HaCaT cells, and, in human keratinocytes, is involved in autophagy regulation through the inhibition of the mTOR signaling pathway by apigenin.

In an example of the present invention, the SDS- or DNCB-induced decrease in protein expression of keratin, involucrin and defensin was increased by an AG extract. In addition, the SDS- or DNCB-induced increase in TNFα expression was normalized by treatment with AG. The expression of PPARδ and AMPK was elevated by the AG extract treatment as compared to SDS or DNCB treatment alone. Furthermore, since the effect of the AG extract was offset by a PPARδ antagonist, it is thought that the effect of AG on keratinocytes is dependent on PPARδ.

Besides PPARδ and AMPK, TRPV4 has also been reported to play an important role in the maintenance or protection of the skin barrier. TRPV4 is generally known as a calcium ion (Ca⁺⁺)-permeable cation transporter, and it responds to mechanical stress such as swelling. In dermatology, TRPV4 protects the skin barrier by strengthening tight junctions and increases keratin synthesis in HaCaT cells treated with baicalein. The role of TRPV4 as a Ca⁺⁺ transporter suggests that it participates in calcium signal transduction in keratinocytes. Ca⁺⁺ generally functions as a signaling molecule in cells and stimulates the expression of biomarkers such as keratin 1/10, involucrin, loricrin and transglutaminase 1, which are involved in keratinocyte differentiation. Moreover, Ca⁺⁺ facilitates the transition of profilaggrin to filaggrin. When the skin barrier is disrupted, the Ca⁺⁺ gradient is lost and the expression of loricrin, filaggrin and involucrin is decreased.

The present invention may provide a composition for preventing, treating or improving an inflammatory skin disease, which contains 3,5-dicaffeoylquinic acid as an active ingredient, and may provide a method for preventing or treating an inflammatory skin disease, which includes a step of administering the 3,5-dicaffeoylquinic acid to a subject.

In the present invention, the subject is not limited as long as it is a mammal having a skin barrier including the stratum corneum. Specifically, it may be human. In the present invention, the inflammatory skin disease may be atopic dermatitis.

In another aspect, the present invention relates to a composition for external application containing said composition, which is in the form of a liquid, an ointment, a cream, a lotion, a spray, a patch, a gel or an aerosol.

The composition of the present invention is administered in a pharmaceutically effective amount. The term “pharmaceutically effective amount” used in The present invention refers to an amount sufficient to treat a disease at a reasonable benefit/risk ratio applicable to medical treatment. An effective dosage level may be determined depending on a variety of factors including the type, disease severity, age and sex of a subject, drug activity, drug sensitivity, administration time, administration route, excretion rate, treatment period, co-administered drugs, and other factors well known in the medical field. The composition of the present invention may be administered either alone or in combination with other therapeutic agents, and the co-administration with conventional therapeutic agents may be carried out sequentially or simultaneously. Single or multiple dosages are possible. It is important to use the composition in the minimum possible amount sufficient to achieve the greatest effect without side effect effects, in consideration of all the factors descried above, and the amount may be determined easily by those skilled in the art.

The pharmaceutical composition of the present invention may contain a pharmaceutically acceptable carrier and may be formulated into a formulation for oral administration such as a powder, a granule, a tablet, a capsule, a suspension, an emulsion, a syrup, aerosol, etc., a formulation for external application, a suppository or a sterile injection solution according to common methods.

The pharmaceutically acceptable carrier includes the ones commonly used in the art, for example, lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, etc., although not being limited thereto. In addition, the pharmaceutical composition of the present invention may contain a diluent or excipient such as a filler, an extender, a binder, a wetting agent, a disintegrant, a surfactant, etc. and other pharmaceutically acceptable additives.

When the pharmaceutical composition of the present invention is prepared into a solid preparation for oral administration such as a tablet, a pill, a powder, a granule, a capsule, etc., the solid preparation may include at least one excipient, e.g., starch, calcium carbonate, sucrose, lactose, gelatin, etc. and a lubricant such as magnesium stearate, talc, etc., although not being limited thereto.

When the pharmaceutical composition of the present invention is prepared into a liquid preparation for oral administration such as a suspension, a solution for internal use, an emulsion, a syrup, etc., it may contain a diluent such as water, liquid paraffin, etc., a wetting agent, a sweetener, an aromatic, a preservative, etc., although not being limited thereto.

When the pharmaceutical composition of the present invention is prepared into a preparation for parenteral administration such as a sterilized aqueous solution, a non-aqueous solution, a suspension, an emulsion, a freeze-dried preparation and a suppository, the non-aqueous solution or suspension may include propylene glycol, polyethylene glycol, vegetable oil such as olive oil, an injectable ester such as ethyl oleate, etc., although not being limited thereto. As a base of the suppository, witepsol, macrogol, Tween 61, cocoa butter, laurin butter, glycerogelatin, etc. may be used, although not being limited thereto.

The pharmaceutical composition of the present invention may be administered orally or parenterally (e.g., via intravenous, subcutaneous, intraperitoneal or topical application) depending on purposes, and the administration dosage may be determined adequately by those skilled in the art although it varies depending on the physical condition and body weight of a patient, the severity of a disease, drug type, administration route and administration time.

The administration dosage of the 3,5-dicaffeoylquinic acid isolated from an Aster glehni (AG) extract contained in the pharmaceutical composition of the present invention may be determined adequately by those skilled in the art although it varies depending on the physical condition, body weight and age of a patient, the severity of a disease, drug type, administration route and administration period. For example, the 3,5-dicaffeoylquinic acid isolated from an Aster glehni (AG) extract may be administered at a daily dosage of 1-2000 mg/kg, specifically 10-2000 mg/kg, and the administration may be made once or several times a day.

In another aspect, the present invention relates to a cosmetic composition for improving skin barrier damage and/or alleviating skin inflammation, which contains 3,5-dicaffeoylquinic acid isolated from the Aster glehni (AG) extract as an active ingredient.

In the present invention, the Aster glehni (AG) extract may be one extracted with one or more extraction solvent selected from a C₁₋₄ alcohol, an aqueous solution containing a C₁₋₄ alcohol, dichloromethane, acetone and an aqueous acetone solution.

In the present invention, the inflammatory skin disease may be atopic dermatitis.

In the present invention, the composition may increase the expression of TRPV (transient receptor potential cation channel subfamily V member 4), PPAR-δ (peroxisome proliferator-activated receptor delta) and AMPK (5′ AMP-activated protein kinase).

In The present invention, the composition may decrease the expression of TNF-α (tumor necrosis factor-alpha).

In the present invention, the term “functional health food” refers to food manufactured or processed with ingredients or components that possess the functionality useful for the body, as defined by the Functional Health Food Act No. 6727, and the “functionality” means gaining useful effect on health purposes by the adjustment of nutrients or the physiological effect, etc. for human body structure and function.

The food composition of the present invention may contain a common food additive, and the appropriateness as the “food additive” is judged according to the standards and guidelines of the Korean Food Additive Code approved by the Korea Food and Drug Administration unless specified otherwise.

The substances listed in the “Korean Food Additive Code” include, for example, chemical substances such as ketones, glycine, potassium citrate, nicotinic acid, cinnamic acid, etc., natural additives such as persimmon color, licorice extract, crystalline cellulose, kaoliang color, guar gum, etc., and mixtures such as sodium L-glutamate, alkali additives for noodles, preservatives, tar color, etc.

The food composition of the present invention is for the purpose of improving skin barrier damage and/or alleviating skin inflammation, and may contain 0.01-95 wt %, specifically 1-80 wt %, of 3,5-dicaffeoylquinic acid isolated from an Aster glehni (AG) extract, based on the total weight of the composition.

In addition, the food composition of the present invention may be prepared and processed into a tablet, a capsule, a powder, a granule, a liquid, a pill, etc. for the purpose of improving skin barrier damage and/or alleviating skin inflammation, particularly for preventing and/or improving atopic dermatitis.

For example, a functional health food in tablet form may be prepared by granulating a mixture of 3,5-dicaffeoylquinic acid isolated from an Aster glehni (AG) extract with an excipient, a binder, a disintegrant or other additives according to a common method and then compressing the mixture after optionally adding a lubricant. In addition, the functional health food in tablet form may contain a flavor enhancer, etc. if necessary, and may be prepared into a suitable coated tablet.

As the functional health food in capsule form, a hard capsule may be prepared by filling a mixture of 3,5-dicaffeoylquinic acid isolated from an Aster glehni (AG) extract and an additive such as an excipient, a granule thereof or a coated granule thereof in a common hard capsule, and a soft capsule may be prepared by filling a mixture of 3,5-dicaffeoylquinic acid isolated from an Aster glehni (AG) extract and an additive such as an excipient in a capsule material such as gelatin, etc. The soft capsule may contain a plasticizer such as glycerin, sorbitol, etc., a colorant, a preservative, etc. if necessary.

A functional health food in pill form may be prepared from a mixture of 3,5-dicaffeoylquinic acid isolated from an Aster glehni (AG) extract, an excipient, a binder, a disintegrant, etc. If necessary, the pill may be coated with white sugar, starch, talc or a suitable material.

A functional health food in granule form may be prepared from a mixture of 3,5-dicaffeoylquinic acid isolated from an Aster glehni (AG) extract, an excipient, a binder, a disintegrant, etc., and may contain a flavorant, a flavor enhancer, etc. if necessary. When the particle size of the functional health food in granule form may contain is tested using No. 12 (1680 μm), No. 14 (1410 μm) and No. 45 (350 μm) sieves, the amount passing through the No. 12 sieve and remaining on the No. 14 sieve may be 5.0% or less, and the amount passing through the 45 sieve may be 15.0% or less, based on the total amount.

The definition of such terms as excipient, binder, disintegrant, lubricant, flavor enhancer, flavorant, etc. is described in literatures known in the art and encompasses those with the same or similar functions.

### MODE FOR INVENTION

Hereinafter, the present invention will be described in detail through examples. However, the following examples are for illustrative purposes only and it will be obvious to those having ordinary knowledge in the art that the scope of The present invention is not limited by the examples.

### Example 1: Preparation of Aster glehni (AG) Extract

Dried leaves of Aster glehni (AG) were purchased in Ulleungdo (N 37° 30′, E 130° 52′) in November 2012 and identified by Emeritus Professor Chang-Soo Yook (Department of Pharmacology, Kyung Hee University, Seoul, Korea). Voucher specimens (971-12A-P) were deposited in the herbarium of the Korea Institute of Science and Technology.

To acquire a methanol-soluble extract, 12 kg of chopped AG was extracted 3 times with 70 L of methanol at room temperature. 2.6 kg of the dried extract residue was suspended in water and then partitioned successively with ethyl acetate. 41.0 g of a residue was obtained by evaporating the ethyl acetate fraction under reduced pressure. The ethyl acetate fraction of AG was analyzed by reverse-phase high-performance liquid chromatography (Waters 1500 Series System) with a 2996 PDA detector (254 nm, Waters, Worcester, Mass., USA).

Separation was performed using a Luna C18 column (5 μm, 250×4.6 mm, Phenomenex, Torrance, Calif., USA) with a sample injection volume of 10 μL at 30° C. The mobile phase was a gradient of acetonitrile and 1% phosphoric acid. The gradient system was as follows: 20% acetonitrile (0 minute), 20% acetonitrile (0-10 minutes), 30% acetonitrile (10-20 minutes), 40% acetonitrile (20-30 minutes), 80% acetonitrile (30-40 minutes) and 100% acetonitrile (40-50 minutes). The flow rate of the mobile phase was 1.0 mL/min. The organic solvents used in the extraction procedure and HPLC analysis were purchased from Sigma-Aldrich (St. Louis, Mo., USA).

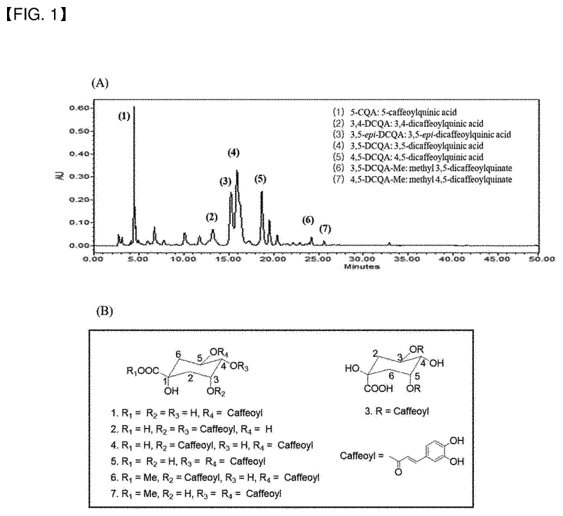

As a result, the ethyl acetate fraction extracted from AG mainly contained 5-caffeoylquinic acid, 3,4-dicaffeoylquinic acid, 3,5-epi-dicaffeoylquinic acid, 3,5-dicaffeoylquinic acid (3,5-DCQA), 4,5-dicaffeoylquinic acid, methyl 3,5-dicaffeoylquinate and methyl 4,5-dicaffeoylquinate (FIG. 1). It was confirmed that 3,5-DCQA was the most abundant among the seven caffeoylquinic acids of the AG ethyl acetate fraction.

In the following examples, the ethyl acetate fraction of the AG methanol extract was used as the AG extract.

### Example 2: Culturing of Keratinocytes (HaCaT)

HaCaT cells (human keratinocytes) were cultured in Dulbecco's modified Eagle's medium (DMEM) containing 10% fetal bovine serum (FBS) and 1% antibiotic-antimycotic solution at 37° C. in a 5% CO₂ incubator. The cell culture medium was replaced with fresh DMEM every 48-72 hours. HaCaT cells within 5-17 passages were plated at a density of 1×10⁴ cells per well in an 8-well chamber slide, or at a density of 1×10⁶ cells per well in a 6-well culture plate in DMEM containing 100% fetal bovine serum and 1% antibiotic-antimycotic solution. The cells were cultured for 24-48 hours in a 5% CO₂ incubator at 37° C. Then, the medium was replaced with DMEM containing 1% fetal bovine serum. Thereafter, the cells were treated for 24 hours with DNCB (5 μM, Sigma-Aldrich, Louis, Mo., USA) or SDS (30 μM, Sigma-Aldrich) along with the AG extract (50 μg) and the PPARδ antagonist GSK0660 (Sigma-Aldrich). In addition, the cells were treated with 1 μM 3,5-dicaffeoylquinic acid (3,5-DCQA). The HaCaT cells were donated by Dr. Sang-Wook Son. All the reagents for cell culture were purchased from Welgene Inc. (Daegu, Korea). The concentrations of all the reagents were final concentrations.

### Example 3: Protein Expression Levels of PPARδ, AMPK, SPTLC2 and TRPV4 in HaCaT Cells Treated with DNCB or SDS Depending on Aster glehni (AG) Extract Treatment

For evaluation of the protein expression levels of PPARδ, AMPK and SPTLC2 in the HaCaT cells treated with SDS or DNCB together with AG, western blot was conducted after treating with the conditions of Example 2.

The concentration of the proteins in the sample was estimated by the Bradford method. 10 μg of the extracted proteins were loaded onto 10% sodium dodecyl sulfate (SDS)-polyacrylamide gel, and protein blotting on nitrocellulose membrane was performed for 90 minutes. The membrane was blocked overnight with 5% skim milk and washed 3 times with TBS-T for 10 minutes. Primary antibodies were allowed to bind to the membrane at room temperature for 2 hours. The primary antibody for PPARδ was purchased from Abcam. The primary antibodies for total and phosphorylated forms of AMPK were purchased from Cell Signaling Technology, Inc. (Danvers, Mass., USA). The primary antibody for serine palmitoyltransferase 2 (SPTLC2) was purchased from Novus and the primary antibody for β-actin was purchased from Santa Cruz Biotechnology, Inc. The dilution conditions for the primary antibodies were as follows: PPARδ, 1:500; AMPK, P-AMPK (at Thr172) and SPTLC2, 1:1000; and β-actin, 1:800. After washing 3 times with TBS-T for 10 minutes, secondary antibodies (Santa Cruz Biotechnology, Inc.) were bound to the membrane at room temperature for 1 hour. The dilution conditions for the antibodies were as follows: anti-rabbit IgG antibodies for PPARδ, AMPK, p-AMPK and SPTLC2, 1:5000; and anti-mouse IgG antibodies for β-actin, 1:5000. After washing 3 times with TBS-T for 10 minutes and washing again once with TBS for another 10 minutes, a solution of a chemiluminescent substrate and an enhancer solution (Bio-Rad, Hercules, Calif., USA) was applied to the membrane to determine the protein expression state. Images were processed manually with Kodak GBX developer and fixer reagents (Carestream Health, Inc., Rochester, N.Y., USA) and were analyzed using the Image J program. β-Actin was used as a internal control group for normalization of the loaded proteins.

As a result, the expression of PPARδ, P-AMPK, SPTLC2 and TRPV4 was increased in the cells treated with the AG extract as compared to the cells treated only with DNCB or SDS. It was also confirmed that the increase in the expression of PPARδ, P-AMPK and SPTLC2 was reduced by treatment with the PPARδ antagonist, GSK0660, along with the AG extract (FIG. 2A and FIG. 2B). From the result, it can be seen that the AG extract activates the expression of TRPV4 in keratinocytes through the sequential regulation of the TRPV4-PPARδ-AMPK pathway.

### Example 4: Expression of Keratin, Involucrin, β-Defensin and TNFα Expression in HaCaT Cells Treated with DNCB or SDS Depending on Aster glehni (AG) Extract Treatment

In order to evaluate the expression of β-defensin 1, a representative β-defensin, together with skin barrier constituents such as keratin and involucrin and the inflammatory cytokine TNFα, immunocytochemistry (ICC) was conducted after treating with the condition of Example 2.

Cells in a chamber slide were fixed with ice-cold methanol for 15 minutes. The fixed cells were treated with a 0.3% hydrogen peroxide (H₂O₂) solution containing 0.3% normal horse serum for 5 minutes to remove peroxidase activity in the cells. After washing with PBS for 5 minutes, the cells were incubated with diluted normal blocking serum for 20 minutes, followed by treatment with diluted primary antibodies for 1 hour. The primary antibodies for involucrin and TNFα were purchased from Novus (Littleton, Colo. 80120, USA). The primary antibody for β-defensin 1 was purchased from Santa Cruz Biotechnology, Inc. (Dallas, Tex., USA), and the primary antibody for pan-keratin was purchased from Abcam (Cambridge, UK). After washing with PBS, the cells were treated with secondary antibodies for 30 minutes. After washing with PBS, the cells were treated with a premixed Vectastain ABC reagent solution for 30 minutes. The cells were washed with PBS and allowed to incubated with a DAB substrate solution until proper color change was observed. After washing with distilled water for 3 minutes, the cells were counter stained with hematoxylin. The cells were washed with distilled water, dried in the air, and finally mounted. The immunocytochemistry kit (including secondary antibodies) was purchased from Vector Laboratories (Burlingame, Calif., USA).

The protein extracts were electrophoresed on 10% polyacrylamide gel and blotted to a nitrocellulose membrane. The nitrocellulose membrane was bound with primary and secondary antibodies sequentially, and then chemiluminescence was exposed to an X-ray film. The density for bands on the X-ray film was analyzed with the Image J program. The HaCaT cells fixed on a slide chamber were immunohistochemically stained with antibodies for keratin, involucrin, defensin and TNFα. Images were taken at 200× magnification, and the densities for the images were analyzed with the Image J program. The results were expressed as mean±SEM. The values were statistically analyzed by unpaired t-test. All the experiments were repeated three times.

As a result, the AG extract increased the expression of the keratin, involucrin and β-defensin 1 proteins, which had been decreased by DNCB and SDS. However, the increased protein expression was offset by the PPARδ antagonist GSK0660. The TNFα expression increased by DNCB and SDS was decreased by the AG extract, and the effect of the AG extract was offset by GSK0660 (FIGS. 3A-3D).

### Example 5: Expression of Keratin, Involucrin, β-Defensin and TNFα in HaCaT Cells

To investigate the effect of TRPV4 and AMPK on the expression of biomarkers related to skin barrier constituents, defense and inflammation, the protein expression for keratin, involucrin, β-defensin and TNFα was evaluated by immunohistochemical staining of HaCaT cells treated with antagonists for TRPV4 and AMPK.

Western blot was conducted for TRPV4, AMPK and PPARδ in HaCaT cells treated with antagonists for TRPV4, PPARδ and AMPK. The HaCaT cells fixed on a slide chamber were immunohistochemically stained with antibodies for keratin, involucrin, defensin and TNFα. Images were taken at 200× magnification, and the densities for the images were analyzed with the Image J program. The protein extracts were electrophoresed on 10% polyacrylamide gel and blotted to a nitrocellulose membrane. The nitrocellulose membrane was bound with primary and secondary antibodies sequentially, and then chemiluminescence was exposed to an X-ray film. The density for bands on the X-ray film was analyzed with the Image J program. The results were expressed as mean±SEM. The values were statistically analyzed by unpaired t-test. All the experiments were repeated three times.

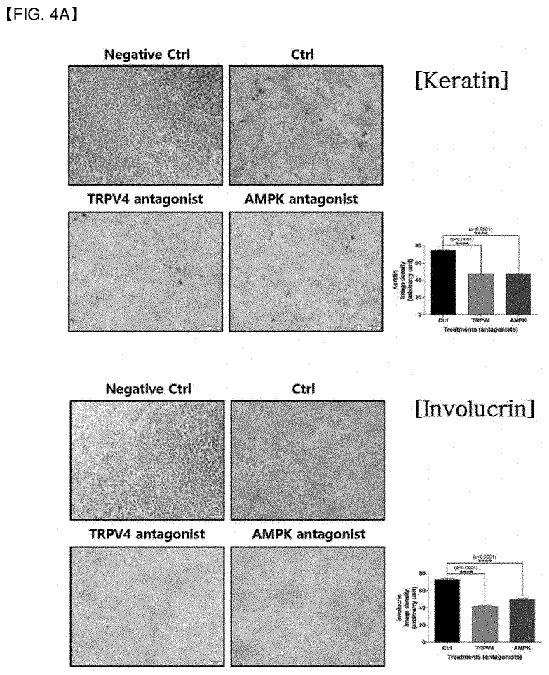

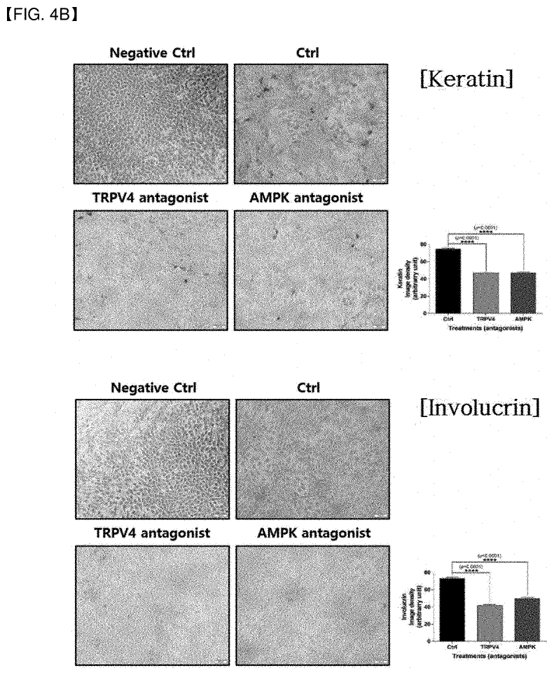

As a result, the protein expression of TNFα was increased by the TRPV4 antagonist or the AMPK antagonist in the HaCaT cells as compared to the control group. However, the protein expression of keratin, involucrin and β-defensin 1 was decreased significantly as compared to the control group under the same condition (FIG. 4A and FIG. 4B).

### Example 6: Correlation Between Protein Expression of TRPV4, PPARδ and AMPK in HaCaT Cells and Antagonists for TRPV4, PPARδ and AMPK

To determine the priority of major regulators which are TRPV4, PPARδ and AMPK, the protein expression level of TRPV4, PPARδ and AMPK in HaCaT cells treated with antagonists for TRPV4, PPARδ or AMPK was measured by western blot.

As a result, in the HaCaT cells treated with the TRPV4 antagonist, all the protein expression levels for TRPV4, PPARδ and p-AMPK were significantly decreased. The PPARδ antagonist treatment lowered the protein expression levels for PPARδ and p-AMPK, but it did not change the TRPV4 protein expression. The AMPK antagonist, compound C, decreased the protein expression only for p-AMPK, but it did not affect the protein expressions for TRPV4 and PPARδ (FIG. 4A and FIG. 4B).

### Example 7: Protein Expression of TRPV4, PPARδ, AMPK and SPTLC2 in HaCaT Cells Treated with 3,5-Dicaffeoylquinic Acid (3,5-DCQA)

In order to measure the protein expression of TRPV4, PPARδ, AMPK and SPTLC2 in HaCaT cells treated with 3,5-dicaffeoylquinic acid, western blot was performed after treating with the condition of Example 2.

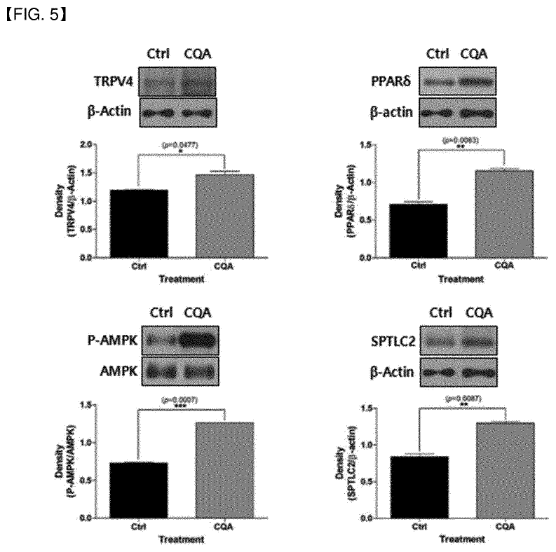

As a result, 3,5-DCQA increased the protein expression of PPARδ, AMPK and SPTLC2 in HaCaT cells as the AG ethyl acetate extract (FIG. 5).

### Example 8: Expression of Keratin, Involucrin, β-Defensin and TNFα in HaCaT Cells Treated with 3,5-Dicaffeoylquinic Acid (3,5-DCQA)

In order to measure the protein expression of keratin, involucrin, β-defensin and TNFα in HaCaT cells treated with 3,5-dicaffeoylquinic acid, immunohistochemical staining was performed after treating with the condition of Example 2.

The protein extracts were electrophoresed on 10% polyacrylamide gel and blotted to a nitrocellulose membrane. The nitrocellulose membrane was bound with primary and secondary antibodies sequentially, and then chemiluminescence was exposed to an X-ray film. The density for bands on the X-ray film was analyzed with the Image J program. The HaCaT cells fixed on a slide chamber were immunohistochemically stained with antibodies for keratin, involucrin, defensin and TNFα. Images were taken at 200× magnification, and the densities for the images were analyzed with the Image J program. The results were expressed as mean±SEM. The values were statistically analyzed by unpaired t-test. All the experiments were repeated three times.

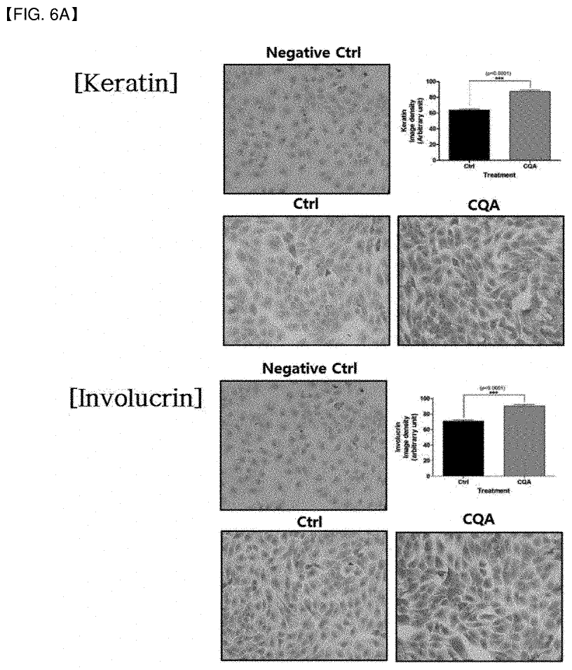

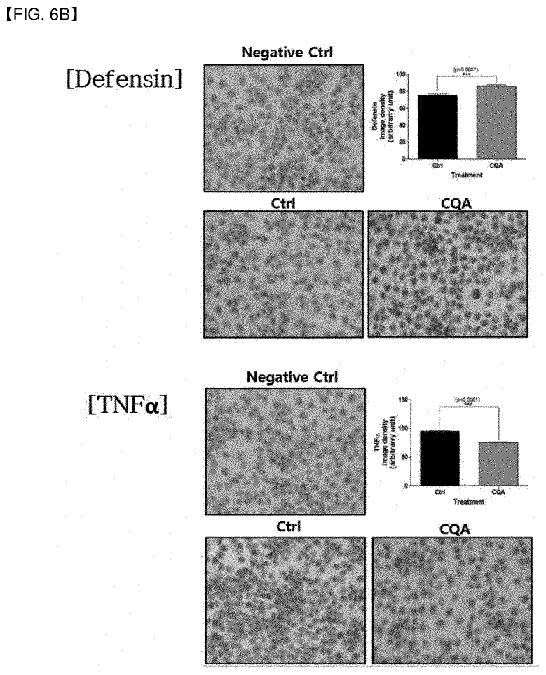

As a result, 3,5-DCQA treatment increased the protein expression of keratin, involucrin and β-defensin 1, which are related to the integrity of the skin barrier, in HaCaT cells, but decreased the expression of TNFα which is related to skin inflammation (FIG. 6A and FIG. 6B).

Since the TRPV4 protein level was increased by the AG extract as compared to the DNCB- or SDS-treated group and the TRPV4 antagonist offset the effect of AG in the keratinocytes, it can be seen that TRPV4 participates in the maintaining of the skin barrier.

The experimental result for the HaCaT cells treated with the antagonists for PPARδ, AMPK and TRPV4 showed that AG can protect keratinocytes through the sequential regulation of the TRPV4→PPARδ→AMPK pathway. In addition, since the 3,5-dicaffeoylquinic acid which is the most abundant among the seven caffeoylquinic acids of the AG extract showed an effect equivalent to that of the ethyl acetate extract in the expressions of the biomarkers related to skin barrier integrity and inflammation in HaCaT cells, it can be seen that the function of the AG extract is attributed to the caffeoylquinic acids.

It was confirmed that the caffeoylquinic acids-rich AG extract can protect the skin barrier of HaCaT keratinocytes treated with SDS or DNCB through the sequential regulation of the TRPV4-PPARδ-AMPK pathway (FIG. 7).

Statistics

Data were presented as mean±SE (standard error of measures).

Statistically significant differences between two groups were calculated by the unpaired t-test using the GraphPad Prism software. A value of p<0.05 was considered significant.

### Preparation Examples: Pharmaceutical Composition and Functional Health Food Composition Containing 3,5-Dicaffeoylquinic Acid as Active Ingredient

### <Preparation Example 1> Preparation of Pharmaceutical Preparations

<1-1> Preparation of Powder

| | |
|---|---|
| 3,5-dicaffeoylquinic acid | 2 g |
| Lactose | 1 g |

A powder was prepared by mixing the above ingredients and filling in an airtight pouch.

<1-2> Preparation of Tablet

| | |
|---|---|
| 3,5-dicaffeoylquinic acid | 100 mg |
| Cornstarch | 100 mg |
| Lactose | 100 mg |
| Magnesium stearate | 2 mg |

A tablet was prepared by mixing the above ingredients and tableting the mixture according to a common method.

<1-3> Preparation of Capsule

| | |
|---|---|
| 3,5-dicaffeoylquinic acid | 100 mg |
| Cornstarch | 100 mg |
| Lactose | 100 mg |
| Magnesium stearate | 2 mg |

A capsule was prepared according to a common method by mixing the above ingredients and filling in a gelatin capsule.

<1-4> Preparation of Pill

| | | |
|---|---|---|
| 3,5-dicaffeoylquinic acid | 1 | g |
| Lactose | 1.5 | g |
| Glycerin | 1 | g |
| Xylitol | 0.5 | g |

A 4-g pill was prepared according to a common method by mixing the above ingredients.

<1-5> Preparation of Granule

| | |
|---|---|
| 3,5-dicaffeoylquinic acid | 150 mg |
| Soybean extract | 50 mg |
| Glucose | 200 mg |
| Starch | 600 mg |

After mixing the above ingredients, adding 100 mg of 30% ethanol and drying at 60° C., the formed granule was filled in a pouch.

### <Preparation Example 2> Preparation of Food

<2-1> Preparation of Wheat Flour Food

After adding 0.5-5.0 parts by weight of the 3,5-dicaffeoylquinic acid of The present invention to 100 parts by weight of wheat flour, bread, cake, cookie, cracker and noodles were prepared using the mixture.

<2-2> Preparation of Soup or Gravy

Soup or gravy for processed meat and noodles for health improvement was prepared by adding 0.1-5.0 parts by weight of the 3,5-dicaffeoylquinic acid of The present invention to 100 parts by weight of soup or gravy 100.

<2-3> Preparation of Ground Beef

Ground beef for health improvement was prepared by adding 10 parts by weight of the 3,5-dicaffeoylquinic acid of The present invention to 100 parts by weight of ground beef 100.

<2-4> Preparation of Dairy Products

After adding 5-10 parts by weight of the 3,5-dicaffeoylquinic acid of The present invention to 100 parts by weight of milk, various dairy products such as butter and ice cream were prepared using the milk.

<2-5> Preparation of Raw Meal Brown rice, barley, glutinous rice and Job's tear were gelatinized, dried and pulverized to 60-mesh powder according to a known method.

Also, black bean, black sesame and perilla seeds were steamed, dried and pulverized to 60-mesh powder according to a known method.

The 3,5-dicaffeoylquinic acid of the present invention was concentrated using a vacuum concentrator, dried with a hot air dryer and then pulverized to 60-mesh powder.

The powders of the grans, seeds and 3,5-dicaffeoylquinic acid prepared above were mixed with the following ratio.

Grains (brown rice 40 wt %, Job's tear 15 wt %, barley 20 wt %),

Seeds (perilla seeds 7 wt %, black bean 8 wt %, black sesame 7 wt %),

3,5-dicaffeoylquinic acid (3 wt %),

Ganoderma lucidum (0.5 wt %),

Rehmannia glutinosa (0.5 wt %)

### <Preparation Example 3> Preparation of Beverage

<3-1> Preparation of Health Drink

After homogeneously mixing 5 g of the 3,5-dicaffeoylquinic acid of the present invention with high-fructose corn syrup (0.5%), oligosaccharide (2%), sugar (2%), table salt (0.5%) and water (75%), the mixture was flash-pasteurized and packaged in a small-volume container such as a glass bottle, a PET bottle, etc.

<3-2> Preparation of Vegetable Juice

A vegetable juice was prepared by adding 5 g of the 3,5-dicaffeoylquinic acid of the present invention to 1,000 mL of tomato or carrot juice.

<3-3> Preparation of Fruit Juice

A fruit juice was prepared by adding 1 g of the 3,5-dicaffeoylquinic acid of the present invention to 1,000 mL of apple or grape juice.

### Preparation Example 2: Preparation of Composition for External Application to Skin and Cosmetic Composition Containing 3,5-Dicaffeoylquinic Acid as Active Ingredient

Although an ointment for external application to skin, a softening lotion, an astringent lotion, a nourishing lotion, a massage cream, an essence and a pack are presented as formulation examples of the present invention, the formulation of the cosmetic composition of the present invention is not limited thereto but may be changed generally by those skilled in the art within the scope of the present invention.

### Formulation Example 1: Ointment for External Application to Skin

An ointment for external application to skin containing 3,5-dicaffeoylquinic acid as described in Table 1 was prepared according to a common method.

TABLE 1

| No. | Ingredients | Contents (wt %) |
|---|---|---|
| 1 | 3,5-dicaffeoylquinic acid | 10 |
| 2 | Diethyl Sebacate | 8 |
| 3 | Spermaceti Wax | 5 |
| 4 | Polyoxyethylene oleyl ether phosphate | 6 |
| 5 | Sodium benzoate | Adequate |
| 6 | Vaseline | Balance |
| 7 | Total | 100 |

### Formulation Example 2: Softening Lotion

TABLE 2

| No. | Ingredients | Contents (wt %) |
|---|---|---|
| 1 | Glycerin | 5.00 |
| 2 | 1,3-Butylene Glycol | 3.00 |
| 3 | PEG 1500 | 1.00 |
| 4 | Allantoin | 0.10 |
| 5 | DL-Panthenol | 0.30 |
| 6 | EDTA-2Na | 0.02 |
| 7 | Benzophenone-9 | 0.04 |
| 8 | Ethanol | 10.00 |
| 9 | Octyldodeceth-16 | 0.20 |
| 10 | Polysorbate 20 | 0.20 |
| 11 | 3,5-dicaffeoylquinic acid | 5.0 |
| 12 | Antiseptic, fragrance and flavor | Trace amount |
| 13 | Distilled water | Balance |

### Formulation Example 3: Astringent Lotion

TABLE 3

| No. | Ingredients | Contents (wt %) |
|---|---|---|
| 1 | Glycerin | 2.00 |
| 2 | 1,3-Butylene Glycol | 2.00 |
| 3 | Allantoin | 0.10 |
| 4 | DL-Panthenol | 0.30 |
| 5 | EDTA-2Na | 0.02 |
| 6 | Benzophenone-9 | 0.04 |
| 7 | Ethanol | 15.00 |
| 8 | Polysorbate 20 | 0.20 |
| 9 | 3,5-dicaffeoylquinic acid | 3.0 |
| 10 | Citric acid | Trace amount |
| 11 | Antiseptic, fragrance and flavor | Trace amount |
| 12 | Distilled water | Balance |

### Formulation Example 4: Nourishing Lotion

TABLE 4

| No. | Ingredients | Contents (wt %) |
|---|---|---|
| 1 | Cetearyl alcohol | 1.00 |
| 2 | Glyceryl stearate | 0.50 |
| 3 | Polysorbate 60 | 1.00 |
| 4 | Sorbitan sesquioleate | 0.30 |
| 5 | Cetyl octanoate | 6.00 |
| 6 | Squalane | 4.00 |
| 7 | Safflower oil | 4.00 |
| 8 | Butylene Glycol | 4.00 |
| 9 | Glycerin | 4.00 |
| 10 | Carbomer | 0.10 |
| 11 | Triethanolamine | 0.10 |
| 12 | 3,5-dicaffeoylquinic acid | 1.00 |
| 13 | Antiseptic, fragrance and flavor | Trace amount |
| 14 | Distilled water | Balance |

### Formulation Example 5: Essence

TABLE 5

| No. | Ingredients | Contents (wt %) |
|---|---|---|
| 1 | Glycerin | 10.00 |
| 2 | Betaine | 5.00 |
| 3 | PEG 1500 | 2.00 |
| 4 | Allantoin | 0.10 |
| 5 | DL-Panthenol | 0.30 |
| 6 | EDTA-2Na | 0.02 |
| 7 | Benzophenone-9 | 0.04 |
| 8 | Hydroxyethylcellulose | 0.10 |
| 9 | Carboxyvinyl polymer | 0.20 |
| 10 | Triethanolamine | 0.18 |
| 11 | Octyldecanol | 0.30 |
| 12 | Octyldodeceth-16 | 0.40 |
| 13 | Ethanol | 6.00 |
| 14 | 3,5-dicaffeoylquinic acid | 5.00 |
| 15 | Antiseptic, fragrance and flavor | Trace amount |
| 16 | Distilled water | Balance |

### Formulation Example 6: Pack

TABLE 6

| No. | Ingredients | Contents (wt %) |
|---|---|---|
| 1 | Polyvinyl alcohol | 15.00 |
| 2 | Cellulose gum | 0.15 |
| 3 | Glycerin | 3.00 |
| 4 | PEG 1500 | 2.00 |
| 5 | Cyclodextrin | 0.15 |
| 6 | DL-Panthenol | 0.30 |
| 7 | Allantoin | 0.10 |
| 8 | Monoammonium glycyrrhizinate | 0.20 |
| 9 | Nicotinamide | 0.40 |
| 10 | Ethanol | 5.00 |
| 11 | PEG 40 hydrogenated castor oil | 0.30 |
| 12 | 3,5-dicaffeoylquinic acid | 1.00 |
| 13 | Antiseptic, fragrance and flavor | Trace amount |
| 14 | Distilled water | Balance |

### Example 9: Skin Barrier Function-Improving Effect of Cosmetic Composition According to the Present Invention

It was investigated whether the 3,5-dicaffeoylquinic acid of the present invention enhances the restoration of damaged skin barrier.

Ten healthy volunteers with normal skin having no dermatological disorder at all were selected as subjects. The skin barrier on the inside forearm was damaged by tape stripping until transepidermal water loss reached around 30 g/m²/h. Then, after evaluating transepidermal water loss (TEWL), 20 μL of the ointment for external application of Preparation Example 2 was applied on the forearm of the volunteers on an area of 9 cm² for 30 minutes, twice a day. The restoration of the skin barrier function was evaluated by measuring the decrease in TEWL every day for 7 days. The recovery rate was measured according to Equation 1.

Br (damage recovery rate)=(1−(Bt=i−Bt=0)/(Bt=d−Bt=0)×100 [Equation 1]

Bt=i=TEWL value at each time after skin barrier damage

Bt=0=TEWL value before skin barrier damage

Bt=d=TEWL value after skin barrier damage

As shown in Table 7, the skin barrier recovery rate of the skin to which a cream containing the 3,5-dicaffeoylquinic acid of the present invention (formulation example) was applied about 19.6% higher 1 day later, and 17.7% higher 3 days later, as compared to a cream not containing the 3,5-dicaffeoylquinic acid. Through this, it was confirmed that the 3,5-dicaffeoylquinic acid of the present invention has an excellent effect of restoring the damaged skin barrier.

TABLE 7

| | Recovery rate (%) | | | |
|---|---|---|---|---|
| | Day 1 | Day 3 | Day 4 | Day 7 |
| Formulation Example (composition | 48.5 | 69 | 83.2 | 95.3 |
| for external application) | | | | |
| Comparative Formulation Example | 28.9 | 51.3 | 68.4 | 82.5 |

While the exemplary embodiments have been shown and described, it will be understood by those skilled in the art that various changes in form and details may be made thereto without departing from the spirit and scope of this disclosure as defined by the appended claims.

### INDUSTRIAL APPLICABILITY

In the present invention, the effect of improving the skin barrier and preventing and treating an inflammatory skin disease of a compound isolated from a natural product was identified. It is expected that the composition of the present invention can be applied to various fields including drugs, quasi-drugs, cosmetics, functional foods, etc. for preventing, treating or improving inflammatory skin diseases by strengthening the physical barrier of skin and alleviating skin inflammation.

1. A method for improving skin barrier damage and/or preventing skin damage, comprising a step of administering 3,5-dicaffeoylquinic acid to a subject.

2. The method for improving skin barrier damage and/or preventing skin damage according to claim 1,
wherein the 3,5-dicaffeoylquinic acid is one isolated from an Aster glehni (AG) extract.

3. The method for improving skin barrier damage and/or preventing skin damage according to claim 2,
wherein the Aster glehni (AG) extract is one extracted with one or more extraction solvent selected from a C1-4 alcohol, an aqueous solution containing a C1-4 alcohol, dichloromethane, acetone and an aqueous acetone solution.

4. The method for improving skin barrier damage and/or preventing skin damage according to claim 3,
wherein the Aster glehni (AG) extract is a fractional extract further fractionated with any one selected from a group consisting of ethyl acetate, hexane, chloroform and butanol.

5. The method for improving skin barrier damage and/or preventing skin damage according to claim 1,
wherein the 3,5-dicaffeoylquinic acid increases the expression of TRPV (transient receptor potential cation channel subfamily V member 4), PPAR-δ (peroxisome proliferator-activated receptor delta) and AMPK (5′ AMP-activated protein kinase).

6. The method for improving skin barrier damage and/or preventing skin damage according to claim 1,
wherein the 3,5-dicaffeoylquinic acid decreases the expression of TNF-α (tumor necrosis factor-alpha).

7. The method for improving skin barrier damage and/or preventing skin damage according to claim 1,
wherein the 3,5-dicaffeoylquinic acid increase thickness of skin barrier.

8. The method for improving skin barrier damage and/or preventing skin damage according to claim 1,
wherein a step of administering is administering 3,5-dicaffeoylquinic acid in the form of a liquid, an ointment, a cream, a lotion, a spray, a patch, a gel or an aerosol to external skin of the subject.

9-13. (canceled)

14. A functional health food composition for improving skin barrier damage, preventing skin damage, and/or alleviating skin inflammation, containing 3,5-dicaffeoylquinic acid as an active ingredient.

15. The functional health food composition for improving skin barrier damage, preventing skin damage, and/or alleviating skin inflammation according to claim 14,
wherein the 3,5-dicaffeoylquinic acid is one isolated from an Aster glehni (AG) extract.

16. The functional health food composition for improving skin barrier damage, preventing skin damage, and/or alleviating skin inflammation according to claim 15,
wherein the Aster glehni (AG) extract is one extracted with one or more extraction solvent selected from a C1-4 alcohol, an aqueous solution containing a C1-4 alcohol, dichloromethane, acetone and an aqueous acetone solution.

17. The functional health food composition for improving skin barrier damage, preventing skin damage, and/or alleviating skin inflammation according to claim 15,
wherein the Aster glehni (AG) extract is a fractional extract further fractionated with any one selected from a group consisting of ethyl acetate, hexane, chloroform and butanol.

18. The functional health food composition for improving skin barrier damage and/or alleviating skin inflammation according to claim 14,
wherein the inflammatory skin disease is atopic dermatitis.

19-20. (canceled)
